(19) United States
(12) Patent Application Publication
(10) Pub. No.: US 2023/0134782 A1
(43) Pub. Date: May 4, 2023

(54) TREATMENT OF SKIN CONDITIONS USING HIGH KRAFFT TEMPERATURE ANIONIC SURFACTANTS

(71) Applicant: ARCUTIS BIOTHERAPEUTICS, INC., Westlake Village, CA (US)

(72) Inventors: David W. OSBORNE, Fort Collins, CO (US); David BERK, Westlake Village, CA (US)

(73) Assignee: ARCUTIS BIOTHERAPEUTICS, INC., Westlake Village, CA (US)

(21) Appl. No.: 17/998,098

(22) PCT Filed: May 6, 2021
(86) PCT No.: PCT/US2021/031144

§ 371 Date: Nov. 7, 2022

Related U.S. Application Data
(60) Provisional application No. 63/021,400, filed on May 7, 2020.

(51) Int. Cl. A61K 31/661; A61K 9/06; A61K 9/00; A61P 17/00; A61K 47/06; A61K 47/10; A61K 47/14; A61K 31/122; A61K 31/52; A61K 31/436; A61K 35/04; A61K 31/519; A61K 31/37; A61K 31/19; A61K 31/513; A61K 31/655

(52) U.S. Cl. CPC A61K 31/661; A61K 9/06; A61K 9/0014; A61P 17/00; A61K 47/06; A61K 47/10; A61K 47/14; A61K 31/122; A61K 31/52; A61K 31/436; A61K 35/04; A61K 31/519; A61K 31/37; A61K 31/19; A61K 31/513; A61K 31/655

(57) ABSTRACT

The present invention is a method and composition for the treatment of skin conditions where the epidermal barrier has decreased function such as when the patient is suffering from eczema, in particular, Atopic Dermatitis. Epidermal barrier function can be significantly improved and the extraction of epidermal lipids can be reduced by using formulations containing high Krafft temperature surfactants, preferably, anionic surfactants.

### FIELD OF THE INVENTION

The invention pertains to the treatment of skin conditions such as eczema where the epidermal barrier is decreased. Epidermal barrier function can be significantly improved by using formulations containing high Krafft temperature anionic surfactants.

### BACKGROUND OF INVENTION

The epidermal barrier has several functions including maintaining water balance, reducing oxidative stress, protecting against foreign substances such as microbes and antigens and protecting against ultraviolet light damage. The entire epidermis is involved in the epidermal barrier but the stratum corneum is mainly responsible for many of these functions. The stratum corneum is made up of several layers of corneocyte cells with intercellular lipid lamellae between the cells. The intercellular lipid lamellae are mainly composed of ceramides, cholesterol, and fatty acids. The cornecytes contain a mixture of small hygroscopic compounds which are involved in the physiological maintenance of hydration in the stratum corneum. These compounds are collectively referred to as natural moisturizing factor (NMF). The epidermal barrier can be compromised by exposure to irritants, improper skin care, low ambient humidity, topical medications, systemic medications, as well as conditions such as atopic dermatitis, rosacea, diabetes, and advanced age. When the epidermal barrier is decreased, proteins and lipids in the stratum corneum can be altered and transepidermal water loss (TEWL) can increase leading to compromised, irritated skin. Epidermal barrier dysfunctions where the epidermal barrier is decreased are treated to control itching, suppress inflammation, and to restore the skin barrier. Epidermal barrier dysfunctions where the epidermal barrier is decreased require different treatments than epidermal barrier dysfunctions which result in hyperproliferative skin diseases such as psoriasis and keratosis. Hyperproliferative skin diseases can be treated with keratolytic agents to remove dead skin cells and reduce scaling. Keratolytic agents should not be used to treat epidermal barrier dysfunctions where the epidermal barrier is decreased as a further reduction in the epidermal barrier is not desirable and such agents will dry out and further irritate the skin.

Emollients such as lotions, creams and ointments are often used as a first line therapy for the local treatment of decreased epidermal barrier function. Emollients provide water and lipids which can help in restoring the epidermal barrier. For emulsions, i.e. emollient creams or lotions, high water content (greater than 20%) is combined with occlusive agents (petrolatum, waxes, oils, silicones) by use of an emulsifier (usually a blend of surfactants) to form a stable topical product. An emollient cream or lotion is a preferred vehicle for medicated topical treatments. Emollient ointments do not necessarily require the addition of a surfactant, but the “greasy feel” is often found to be objectionable and thus patients prefer to apply a cream or lotion.

The interplay between surfactants and the stratum corneum, specifically the lipids of the stratum corneum, has been used to explain why some surfactants are highly irritating to the skin while others appear relatively inert. In broadest terms, topically applied surfactants can alter the barrier properties of the stratum corneum (SC) which allows a greater influx of potential irritants. The irritant may be the surfactant itself, another excipient from the topical product, a degradant or contaminant carried into the topical product as a trace impurity of the active/excipient, or an environmental irritant that inadvertently comes in contact with the same anatomical site previously dosed with the surfactant-based topical product. The likelihood of surfactant induced skin irritation dramatically increases when treating conditions such as atopic dermatitis (AD) which is mechanistically linked to decreased skin barrier function (Peter M. Elias, Yutaka Hatano and Mary L. Williams. Basis for the barrier abnormality in atopic dermatitis: Outside-inside-outside pathogenic mechanisms. J Allergy Clin Immunol. 2008 June; 121(6): 1337-1343. doi: 10.1016/j.jaci.2008.01.022).

More specifically, three conceptual surfactant-stratum corneum lipid interactions alter the barrier properties of the skin: 1) surfactant monomers adsorb onto the surface of the SC and increase skin wettability, 2) surfactants mix with and disorganize the bilayer structured epidermal lipids and 3) surfactant micelles solubilize/extract lipids from the SC (Lemery E, Briancon S, Chevalier Y, Oddos T, Gohier A, Boyron O, Bolzinger M A. Surfactants have multi-fold effects on skin barrier function. Eur J Dermatol 2015; 25(5): 424-35 doi: 10.1684/ejd.2015.2587). Monomers of the anionic surfactant sodium dodecyl sulfate (SDS) very effectively adsorb, mix and disorganize skin lipids and SDS micelles effectively extract epidermal lipids which results in aqueous solutions of SDS being highly irritating to skin. It should be noted that an anionic surfactant will mix with and disorganize the bilayer structured epidermal lipids (step 2) of epidermal barrier compromised skin much faster and more completely than with normal skin.

Since first line therapy of dry, itchy skin in general is topical application of emollient creams or lotions, the application of surfactants to barrier compromised skin cannot be avoided. Since emollient emulsions are the preferred topical treatment, formulators endeavor to use surfactants having low irritation potential. Certain nonionic surfactants are too bulky to mix with the bilayer structured epidermal lipids (mechanistic step 2) and are known to be very mild. Specifically, formulators of emollient emulsions prefer nonionic surfactants that have large PEG headgroups which inhibit the penetration of these surfactants into the SC lipid matrix. Such surfactants include poly(oxyethylene)-20 sorbitan laurate, PEG-12 dimethicone (conclusion of the Lemery et. al. paper) and ceteth-20.

Surfactant induced extraction of epidermal lipids, the third mechanistic step in surfactant induced skin irritation requires further description. When an emollient cream or lotion is rubbed into barrier compromised skin, water from the formulation will hydrate the SC and occlusive agents will “trap” water in the SC to temporarily restore barrier function and provide relief from skin irritation. If the emollient is applied after bathing, skin moisturization will be enhanced because water retained on the skin combined with water from the emulsion will be trapped by the occlusive agents to prolong restoration of the skin barrier and irritation relief. In time, the occlusive agents will wear off and the hydrating water of the SC will be lost; then skin irritation will return. The duration of benefit for an emollient cream or lotion depends on various factors, but the relative humidity of the air surrounding the skin is a primary factor. An emollient may provide relief for a few hours in a dry environment compared to 6-8 hours in a more humid environment. If the emollient emulsion contains barrier restorative lipids, e.g. ceramides, in addition to occlusive agents the duration of the benefit can be significantly extended. A physically stable topical product that contains similar amounts of water and lipids requires the formulation to contain surfactants. If the surfactants used in the emollient formulation mix well with the epidermal lipids of the stratum corneum, then the topical product can potentially extract epidermal lipids and decrease the barrier function of the skin over time. This extraction step occurs when surfactant micelles form to solubilize the epidermal lipids and complete the extraction process. Epidermal lipid extraction efficiency can be directly related to the extent of skin barrier compromise and the potential to irritate the skin.

Mechanistically, surfactant induced extraction of epidermal lipids occurs in the presence of micelles. When dissolved in water, both anionic and nonionic surfactant monomers associate to form micelles over a specific concentration and temperature range. Once the concentration of surfactant is above the critical micellization concentration (CMC), the physical properties of surfactant solutions dramatically change, most notably in the ability of this aqueous solution to solubilize significant amounts of lipid. Nonionic surfactants almost always spontaneously form micelles below room temperature. Anionic surfactants differ from nonionic surfactants in that the formation of micelles may require warming the solution above ambient temperatures in addition to having surfactant concentrations above the CMC. The minimum temperature required for an anionic surfactant to form micelles is known as the Krafft temperature (named after Friedrich Krafft for his work on soaps as colloids 1894-1900). Below the Krafft temperature, increasing the concentration of the surfactant above the CMC results in sedimented solid surfactant rather than the formation of micelles. Thus, the Krafft temperature is the temperature at which the surfactant dissolves which is affected by the concentration. The Krafft temperature for a specific anionic surfactant can either increase or decrease up to a few degrees Celsius as the concentration of the surfactant is increased beyond the CMC.

Micelles can only form if enough water is present for the surfactant to remain in the specific concentration and temperature range. While an excess of a 2% surfactant solution can be held against excised human skin for 20 hours in a laboratory setting, most people will experience surfactant induced lipid extraction only while bathing, showering or swimming. The most common “real life” scenario for significant surfactant induced extraction of epidermal lipids is during a long soak in a hot bath.

The acceptable water temperature range for bathing adults is 38 to 43 degrees Celsius (109.4° F.) [Alberta Health Services Procedure for Safe Bathing Temperatures and Frequency, effective date Dec. 2, 2019; extranet.ahsnet.ca/teams/policydocuments/1/clp-provincial-sh-safe-bath-temps-procedure.pdf]. If 43° C. is the highest safe bath temperature, then any surfactant having a Kraftt Temperature of 44° C. or higher would not be able to extract epidermal lipids. A topical emulsion containing anionic emulsifiers having Krafft Temperatures at or above 44° C. can be safely applied to patients having barrier compromised skin without making their skin condition (for example atopic dermatitis) worse. Therefore, high Krafft Temperature emulsifiers such as blends of the alkyl phosphates ceteth-10 phosphate (TK=53° C.) and dicethyl phosphate (TK=58° C.) would significantly improve the epidermal barrier function of patients treated with moisturizing topical formulations. Formulating with emulsifiers having higher Krafft temperatures than the temperature of scalding water does not provide more benefit to patients because no one will intentionally bathe in scalding water. According to the Consumer Products Safety Commission [.accuratebuilding.com/services/legal/charts/hot_water_burn_scalding_graph] adults will suffer third-degree burns if exposed to 130° F. (54.4° C.) water for thirty seconds.

Thus, the treatment of barrier compromised skin with emollient creams or lotions containing surfactants that extract epidermal lipids can induce a cycle of diminished efficacy when the treatment is repeatedly administered. For example, if a patient has reduced epidermal barrier function and presents with atopic dermatitis (AD), an emollient cream (with or without a pharmaceutical active ingredient) could be provided with instructions to use twice daily with application promptly after bathing (before the skin dries out). The emollient cream restores the skin barrier for 10-12 hours giving the patient relief from their AD symptoms for most of the day. Assuming that the patient baths daily, the surfactant that has mixed with the epidermal lipids during the two applications in the previous 24-hours, forms micelles in the bath, solubilizes and extracts epidermal lipids and significantly reduces the skin barrier of the patient. Emollient cream is applied promptly after the bath, restoring the skin barrier. This daily cycle is repeated for four weeks or more. The patient experiences diminished efficacy, possibly 10% or 15% net improvement of their AD symptoms, because the surfactant used forms micelles during bathing and extracts epidermal lipid. If a surfactant was used that could not form micelles, then efficacy would not be diminished and the emollient cream would have maximum efficacy with possibly a 50% net improvement of AD symptoms. This emollient cream would also be an optimal vehicle for addition of an active pharmaceutical ingredient that could provide even greater improvement of AD symptoms.

A need exists for an emollient emulsion which does not extract epidermal lipids and thus does not result in diminished clinical efficacy over time.

### SUMMARY OF THE INVENTION

In accordance with the present invention, it has been discovered that formulations which include high Krafft temperature anionic surfactants reduce the extraction of epidermal lipids and increase epidermal barrier function. Improving epidermal barrier function leads to reduced abnormal desquamation, improvement in elasticity, and reduced skin rigidity resulting in less skin irritation and increased skin hydration.

### BRIEF DESCRIPTION OF THE DRAWINGS

FIG. 1 shows the results of treating excised skin with a high Krafft temperature formulation and a low Krafft temperature formulation. Treatment with the cream formulation containing high Krafft temperature phosphate surfactants (Krafft temperature 53° C., Formulation 2 from EXAMPLE 2) did not result in ceramide extraction. Treatment with the cream containing the low Krafft temperature sodium cetostearyl sulfate surfactant (Elidel Cream Vehicle, Formulation 5 from EXAMPLE 2) was most efficient in extracting ceramides from human skin. A greater quantity of ceramides were extracted from Formulation 5 treated skin after 3 warm water washes than from the 4% sodium lauryl sulfate positive control.

### DETAILED DESCRIPTION OF THE INVENTION

Epidermal barrier compromised skin can be treated using emollient emulsions containing one or more high Krafft temperature anionic surfactants without decreased clinical efficacy over time. The surfactants emulsify the composition and help wet the surface of any actives or excipients in the formulation. As used herein the term “surfactant” means an amphiphile (a molecule possessing both polar and nonpolar regions which are covalently bound) capable of reducing the surface tension of water and/or the interfacial tension between water and an immisicible liquid. Any anionic surfactant with a Krafft temperature above 48° C. can be used in the present invention. The Krafft point of an anionic surfactant can be determined using methods known in the art, for example, see Li, et al., “Property Prediction on Surfactant by Quantitative Structure-Property Relationship: Krafft Point and Cloud Point”, Journal of Dispersion Science and Technology, 26: 799-808, 2005. Such surfactants may include but are not limited to alkyl aryl sodium sulfonate, ammonium lauryl sulfate, cocamide ether sulfate, cocamine oxide, coco betaine, coco diethanolamide, coco monoethanolamide, coco-caprylate/caprate, disodium cocoamphodiacetate, disodium laureth sulfosuccinate, disodium lauryl sulfoacetate, disodium lauryl sulfosuccinate, disodium oleamido monoethanolamine sulfosuccinate, docusate sodium, sodium dodecylbenzenesulfonate, sodium palmitate, sodium hexadecyl sulfonate, sodium stearyl sulfate, sodium stearate, sodium xylene sulfonate, potassium cetyl phosphate, potassium C9-15 alkyl phosphate, potassium C11-15 alkyl phosphate, potassium C12-13 alkyl phosphate, potassium C12-14 alkyl phosphate, potassium lauryl phosphate, C8-10 alkyl ethyl phosphate, C9-15 alkyl phosphate, C20-22 alkyl phosphate, castor oil phosphate, ceteth-10 phosphate, cetheth-20 phosphate, ceteth-8 phosphate, cetearyl phosphate, cetyl phosphate, dimethicone PEG-7 phosphate, disodium lauryl phosphate, disodium oleyl phosphate, lauryl phosphate, myristyl phosphate, octyldecyl phosphate, oleth-10 phosphate, oleth-5 phosphate, oleth-3 phosphate, oleyl ethyl phosphate oleyl phosphate, PEG-26-PPG-30 phosphate, PPG-5 ceteareth-10 phosphate, PPG-5 ceteth-10 phosphate, sodium lauryl phosphate, sodium laureth-4 phosphate, steartyl phosphate, DEA-cetyl phosphate, DEA-oleth-10 phosphate, DEA-oleth-3 phosphate, DEA-C8-C18 perfluoroalkylethyl phosphate, dicetyl phosphate, dilaureth-10 phosphate, dimyristyl phosphate, dioleyl phosphate, tricetyl phosphate, triceteareth-4 phosphate, trilaureth-4 phosphate, trilauryl phosphate, triolyeyl phosphate and tristearyl phosphate.

Commonly Used Anionic Surfactants and Their Krafft Temperatures

| | Krafft Temperature |
|---|---|
| Surfactant | ° C. |
| C₁₀H₂₁SO₃_{^{−}} Na⁺ Sodium decyl sulfonate | 22.5 |
| C₁₂H₂₅SO₃_{^{−}} Na⁺ Sodium dodecyl sulfonate | 38.0 |
| C₁₄H₂₉SO₃_{^{−}} Na⁺ Sodium myristyl* sulfonate | 48.0 |
| C₁₆H₃₃SO₃_{^{−}} Na⁺ Sodium hexadecyl sulfonate | 57.0 |
| C₁₀OSO₃_{^{−}} Na⁺ Sodium decyl sulfate | 8.0 |
| C₁₂H₂₅OSO₃_{^{−}} Na⁺ Sodium lauryl sulfate (SLS) Sodium dodecyl sulfate (SDS) | 16.0 |
| C₁₄H₂₉OSO₃_{^{−}} Na⁺ Sodium myristyl sulfate | 30.0 |
| C₁₆H₃₃OSO₃_{^{−}} Na⁺ Sodium cetyl sulfate | 45.0 |
| C₁₈H₃₇OSO₃_{^{−}} Na⁺ Sodium stearyl sulfate | 56.0 |
| C₁₂H₂₅(OCH₂CH₂)OSO3− Na+ Sodium laureth sulfate (SLES) | <0 |
| C₁₂H₂₅OSO₃_{^{−}} NH₄_{⁺} Ammonium lauryl sulfate (ALS) | 10 |
| C₁₂COO⁻Na⁺ Sodium laurate Dodecanoic acid sodium salt | 21.5 |
| C₁₄COO⁻Na⁺ Sodium myristate Tetradecanoic acid, sodium salt | 39 |
| C₁₆COO⁻Na⁺ Sodium palmitate Hexadecenoic acid, sodium salt | 69 |
| C₁₈COO⁻Na⁺ Sodium stearate Octadecanoic acid, sodium salt | 71 |
| C₁₂H₂₅(C₆H₄)SO₃_{^{−}}Na⁺ Sodium dodecylbenzenesulfonate | 52 |
| C₁₂OPO₃_{^{−}} Na⁺ Sodium lauryl phosphate Sodium dodecyl phosphate (SDP) | 31.5 |
| Ceteth - 10 phosphate | 53 |
| Dicetyl phosphate | 58 |
| Sodium cetostearyl sulfate Blend of cetyl and stearyl sulfate | 41 |

*sodium tetradecyl sulfate is a commonly used (but incorrect) synonym for 7-ethyl-2-methyl-4-undecanyl sulfate sodium which is an anionic surfactant sclerosing agent.

In a preferred embodiment, the emulsifier blend of cetearyl alcohol (CAS 67762 30 0), dicetyl phosphate (CAS 2197 63 9) and ceteth-10 phosphate (CAS 50643-20-4) which is manufactured by Croda under the tradename CRODAFOS™ CES, is used. This commercially available emulsifier blend is a self-emulsifying wax that is predominately the waxy material cetearyl alcohol (which is a mixture cetyl alcohol (C₁₆H₃₄O) and stearyl alcohol (C₁₈H₃₈O)) combined with 10-20% dicetyl phosphate and 10-20% ceteth-10 phosphate. Self-emulsifying waxes form an emulsion when blended with water. When CRODAFOS™ CES is added to water it spontaneously forms an emulsion having a pH of about 3. Agents which adjust the pH can be added to increase or decrease the pH to the desired value. The pH of the formulation can be adjusted depending on the optimal pH of the components. The pH should be between 3.5-9.0, preferably between 4.0-8.0.

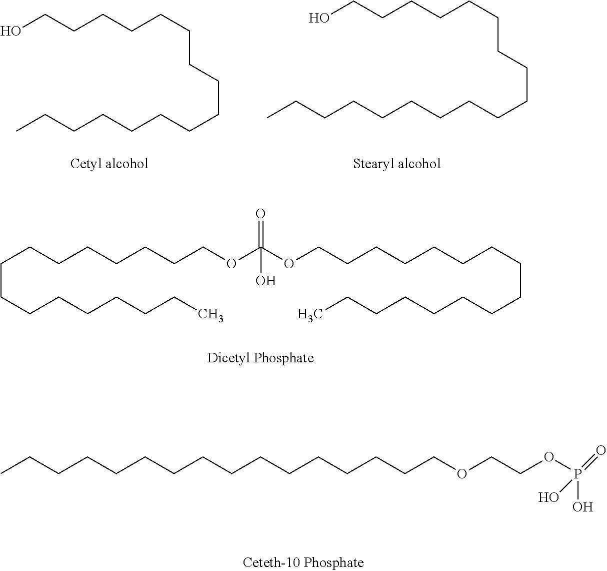

Preferably, the compositions according to the present invention are in one of the following forms:

An oil-in-water emulsion: The product may be an emulsion comprising a discrete phase of a hydrophobic component and a continuous aqueous phase that includes water and optionally one or more polar hydrophilic excipients as well as solvents, co-solvents, salts, surfactants, emulsifiers, and other components. These emulsions may include water-soluble or water-swellable polymers that help to stabilize the emulsion.

A water-in-oil emulsion: The compositions may be an emulsion that includes a continuous phase of a hydrophobic component and an aqueous phase that includes water and optionally one or more polar hydrophilic carrier(s) as well as salts or other components. These emulsions may include oil-soluble or oil-swellable polymers as well as one or more emulsifier(s) to help to stabilize the emulsion.

A hydrophilic or hydrophobic ointment: The compositions are formulated with a hydrophobic base (e.g. petrolatum, thickened or gelled water insoluble oils, and the like) and optionally having a minor amount of a water soluble phase. Hydrophilic ointments generally contain one or more surfactants or wetting agents

A microemulsion: These are clear, thermodynamically stable isotropic liquid systems that contain oil, water and surfactants, frequently in combination with a cosurfactant. Microemulsions may be water continuous, oil continuous or bicontinuous mixtures. The formulations may optionally also contain water up to 60% by weight. Higher levels may be suitable in some compositions. Classes of cosurfactants include short-chain alcohols, alkane diols and triols, polyethylene glycols and glycol ethers, pyrrolidine derivatives, bile salts, sorbitan fatty acid esters and polyoxyethylene sorbitan fatty acid esters. Suitable hydrophilic components for use in a microemulsion include one or more glycols such as polyols such as glycerin, propylene glycol, butylene glycols, polyethylene glycols (PEG), random or block copolymers of ethylene oxide, propylene oxide, and/or butylene oxide, polyalkoxylated surfactants having one or more hydrophobic moieties per molecule, silicone copolyols, blend of ceteareth-6 and stearyl alcohol as well as combinations thereof, and the like.

An aerosol foam or spray: The product may be an alcohol/solvent based solution containing an emulsifying wax or an emulsion comprising a discrete phase of a hydrophobic component and a continuous aqueous phase that includes water and optionally one or more polar hydrophilic excipients as well as solvents, co-solvents, surfactants, emulsifiers, and other components. These solvent or emulsion foam concentrates may include water-soluble or water-swellable polymers that help to stabilize the emulsion and corrosion inhibitors to improve compatibility between the formulation and the package. A hydrocarbon, hydrochlorofluorocarbon (HCFC) or chlorofluorocarbon (CFC) aerosol propellant can be added to the solvent or emulsion foam concentrate in packaging designed to maintain pressure until the foam or spray product is dispensed for application.

### Solvents

Compositions according to the present invention may include one or more solvents or co-solvents which modify skin permeation or the activity of other excipients contained in the formulation. Solvents include but are not limited to ethanol, benzyl alcohol, butyl alcohol, diethyl sebacate, diethylene glycol monoethyl ether, diisopropyl adipate, dimethyl sulfoxide, ethyl acetate, isopropyl alcohol, isopropyl isostearate, isopropyl myristate, oleyl alcohol, polyethylene glycol, glycerol, propylene glycol and SD alcohol.

### Moisturizers

Compositions according to the present invention may include additional moisturizers to increase the level of hydration. The moisturizer can be a hydrophilic material including humectants or it can be a hydrophobic material including emollients. Suitable moisturizers include but are not limited to: 1,2,6-hexanetriol, 2-ethyl-1,6-hexanediol, butylene glycol, glycerin, polyethylene glycol 200-8000, butyl stearate, cetostearyl alcohol, cetyl alcohol, cetyl esters wax, cetyl palmitate, cocoa butter, coconut oil, cyclomethicone, dimethicone, docosanol, ethylhexyl hydroxystearate, fatty acids, glyceryl isostearate, glyceryl laurate, glyceryl monostearate, glyceryl oleate, glyceryl palmitate, glycol distearate, glycol stearate, isostearic acid, isostearyl alcohol, lanolin, mineral oil, limonene, medium-chain triglycerides, menthol, myristyl alcohol, octyldodecanol, oleic acid, oleyl alcohol, oleyl oleate, olive oil, paraffin, peanut oil, petrolatum, Plastibase-50W, and stearyl alcohol.

### Polymers and Thickeners

For certain applications, it may be desirable to formulate a product that is thickened with soluble, swellable, or insoluble organic polymeric thickeners such as natural and synthetic polymers or inorganic thickeners such as acrylates copolymer, carbomer 1382, carbomer copolymer type B, carbomer homopolymer type A, carbomer homopolymer type B, carbomer homopolymer type C, acrylamide/sodium acryloyldimethyl taurate copolymer, carboxy vinyl copolymer, carboxymethylcellulose, carboxypolymethylene, carrageenan, guar gum, hydroxyethyl cellulose, hydroxypropyl cellulose, microcrystalline wax, and methylcellulose,

### Additional Components

Compositions according to the present invention may be formulated with additional components such as fillers, carriers and excipients conventionally found in cosmetic and pharmaceutical topical products. Additional components including but not limited to antifoaming agents, preservatives (e.g. p-hydroxybenzoic esters, benzyl alcohol, phenylmercury salts, chlorocresol), antioxidants, sequestering agents, stabilizers, buffers, pH adjusting agents (preferably agents which result in an acidic pH, including but not limited to gluconolatone, citric acid, lactic acid, and alpha hydroxyacids), skin penetration enhancers, skin protectants (including but not limited to petrolatum, paraffin wax, dimethicone, glyceryl monoisostearate, isopropyl isostearate, isostearyl isostearate, cetyl alcohol, potassium cetyl phosphate, cetyl behenate and behenic acid), chelating agents, film formers, dyes, pigments, diluents, bulking agents, fragrances, aerosol producing agents and other excipients to improve the stability or aesthetics, may be added to the composition. Though alcohol is known to irritate and extract water and lipids from the skin, alcohol can be included in formulations which include high Krafft temperature surfactants in view of the improvement in epidermal barrier function. Alcohol can be included to improve the solubility and to increase the absorption of active pharmaceutical agents.

Compositions according to the present invention may be formulated with or without pharmaceutically active agents depending on the condition being treated. The additional active agents include but are not limited to Anthralin (dithranol), Azathioprine, Tacrolimus, Tapinarof, Coal tar, Methotrexate, Methoxsalen, Ammonium lactate, 5-fluorouracil, Propylthouracil, 6-thioguanine, Sulfasalazine, Mycophenolate mofetil, Fumaric acid esters, Corticosteroids (e.g. Aclometasone, Amcinonide, Betamethasone, Clobetasol, Clocotolone, Mometasone, Triamcinolone, Fluocinolone, Fluocinonide, Flurandrenolide, Diflorasone, Desonide, Desoximetasone, Dexamethasone, Halcinonide, Halobetasol, Hydrocortisone, Methylprednisolone, Prednicarbate, Prednisone), Corticotropin, Vitamin D analogues (e.g. calcipotriene, calcitriol), Acitretin, Tazarotene, Cyclosporine, Resorcinol, Colchicine, Adalimumab, Ustekinumab, Infliximab, phosphodiesterase-4 inhibitors (PDE-4 inhibitors) such as Roflumilast, and antibiotics (e.g. erythromycin, ciprofloxacin, metronidazole).

### Administration and Dosage

The compositions according to the present invention can be administered by any suitable administration route including but not limited to cutaneously (topically), transdermally, and mucosally (e.g. sublingual, buccal, nasally). In a preferred embodiment, the composition is administered topically.

Suitable pharmaceutical dosage forms include but are not limited to emulsions, creams, lotions, foams, microemulsions and nanoemulsions.

The composition can be administered one or more times per day, preferably the composition is administered 1-2 times per day.

The composition can be used in veterinary and in human medicine for the treatment of all diseases and conditions associated with epidermal barrier dysfunction, such as proliferative, inflammatory and allergic dermatoses. Such dermatoses include but are not limited to Inflamed Keratinization Disorders such as atopic dermatitis, psoriasis (vulgaris), eczema, acne, Lichen simplex, sunburn, pruritus, seborrheic dermatitis, Darier disease, Hailey-Hailey disease, hypertrophic scars, discoid lupus erythematosus, and pyodermias. In a preferred embodiment, the dermatoses to be treated is atopic dermatitis.

The following examples are provided to enable those of ordinary skill in the art to make and use the methods and compositions of the invention. These examples are not intended to limit the scope of what the inventor regards as the invention. Additional advantages and modifications will be readily apparent to those skilled in the art.

### Example 1

Creams were prepared according to the following formulations.

### Formulation 1

| | | |
|---|---|---|
| White Petrolatum | 10.0% | w/w |
| Isopropyl Palmitate | 5.0% | w/w |
| Crodafos CES | 10.0% | w/w |
| Diethylene glycol monoethyl ether (Transcutol P) | 25% | w/w |
| Methylparaben | 0.2% | w/w |
| Propylparaben | 0.05% | w/w |

| | |
|---|---|
| Purified Water | q.s. ad 100 (49.75%) |

### Formulation 2

| | | |
|---|---|---|
| White Petrolatum | 10.0% | w/w |
| Isopropyl Palmitate | 5.0% | w/w |
| Crodafos CES | 10.0% | w/w |
| Hexylene glycol | 2.0% | w/w |
| Diethylene glycol monoethyl ether (Transcutol P) | 25.0% | w/w |
| Methylparaben | 0.2% | w/w |
| Propylparaben | 0.05% | w/w |

| | |
|---|---|
| Purified Water | q.s. ad 100 (47.75%) |

### Formulation 3

| | | |
|---|---|---|
| White Petrolatum | 10.0% | w/w |
| Isopropyl Palmitate | 5.0% | w/w |
| Sodium Dodecyl Sulfate | 2.0% | w/w |
| Cetearyl Alcohol | 8.0% | w/w |
| Hexylene glycol | 2.0% | w/w |
| Diethylene glycol monoethyl ether (Transcutol P) | 25.0% | w/w |
| Methylparaben | 0.2% | w/w |
| Propylparaben | 0.05% | w/w |

| | |
|---|---|
| Purified Water | q.s. ad 100 (47.75%) |

Formulation 4 (U.S. Pat. No. 10,195,160—Formulation for Tapinarof 2b in Table 1)

| | | |
|---|---|---|
| Medium Chain Triglycerides | 10.0% | w/w |
| Steareth-2 | 1.8% | w/w |
| Steareth-20 | 1.1% | w/w |
| Polysorbate 80 | 1.5% | w/w |
| Propylene glycol | 10.0% | w/w |
| Diethylene glycol monoethyl ether (Transcutol P) | 2.0% | w/w |
| Benzoic acid | 0.25% | w/w |
| Butylated hydroxytoluene | 0.1% | w/w |
| Disodium ethylene diamine tetraacetic acid | 0.1% | w/w |
| Citrate/citric acid buffer | 0.27% | w/w |

| | |
|---|---|
| Purified Water | q.s. ad 100 (64.68%) |

### Formulation 5 (Formulation for Elidel Cream Vehicle Example 14 EP 0 786986)

| | | |
|---|---|---|
| Mono- and di-glycerides | 2.0% | w/w |
| Medium-chain triglycerides | 15.0% | w/w |
| Sodium cetostearyl sulphate | 1.0% | w/w |
| Propylene glycol | 5.0% | w/w |
| Cetyl alcohol | 4.0% | w/w |
| Benzyl alcohol | 1.0% | w/w |
| Stearyl alcohol | 4.0% | w/w |
| Oleyl alcohol | 10.0% | w/w |
| Citrate/citric acid buffer | 0.05% | w/w |

| | |
|---|---|
| 10% NaOH or 10% HCl Solution | as needed for pH = 5.3 + 0.3 |
| Purified Water | q.s. ad 100 (57.95%) |

### Example 2

0.0012 grams of ceteth-10 phosphate (Moravek Lot 671-144-000-A-20190821-JHO) was weighed into a 20 mL glass scintillation vial. 10.0113 grams of distilled water was added to the scintillation vial and the vial was tightly capped and placed in a water bath. The temperature was gradually increased from 36.0° C. to 56.0° C. After equilibrating at 52.5° C. for 150 minutes the ceteth-10 phosphate had not dissolved and the sample did not froth when vigorously shaken. The surfactant remained as waxy particles sedimented on the bottom of the vial. After equilibration at 53.0° C. for 435 minutes, ceteth-10 phosphate had dissolved and the sample frothed when shaken. The Krafft temperature of a 0.012% ceteth-10 phosphate aqueous solution was determined to be 53.0° C.

0.0019 grams of dicetyl phosphate (Sigma dihexadecyl phosphate lot STBH2863) was weighed into a 20 mL glass scintillation vial. 11.2262 grams of distilled water was added to the scintillation vial and the vial was tightly capped and placed in a water bath. The temperature was gradually increased from 51.0° C. to 65.0° C. After equilibrating at 57° C. for 120 minutes the dicetyl phosphate had not dissolved and the sample did not froth when vigorously shaken. After equilibration at 58.0° C. for 450 minutes, dicetyl phosphate had dissolved and the sample frothed when shaken. The Krafft temperature of a 0.017% dicetyl phosphate aqueous solution was determined to be 58.0° C. 0.0024 grams of sodium cetostearyl sulfate (BASF Lanette E Granules Lot 0021826181) was weighed into a 20 mL glass scintillation vial. 17.0763 grams of distilled water was added to the scintillation vial and the vial was tightly capped and placed in a water bath. The temperature was gradually increased from 35.0° C. to 42.5° C. After equilibrating at 40.0° C. for 805 minutes the sodium cetostearyl sulfate had not dissolved and the sample slightly frothed when vigorously shaken. After equilibration at 42.5° C. for 365 minutes, sodium cetostearyl sulfate had dissolved and the sample frothed when shaken. The Krafft temperature of a 0.014% sodium cetostearyl sulfate aqueous solution was determined to be 41.0° C.

### Example 3

The ability of cream formulations containing emulsifiers having a range of Krafft temperatures, to extract epidermal lipids can be determined using excised human cadaver skin dermatomed to a target thickness of 500 microns. Excised human skin was obtained frozen from a US tissue bank and stored at −20° C. until use. The skin was loaded onto vertical Franz cells with a diameter of 8 mm having a 0.503 cm² extraction area and a receptor chamber filled with 3.0 ml of 4% BSA in water containing 0.01% gentamicin sulfate thermostated at 32° C. (receptor solution). Using a positive displacement pipette, a 5-microliter dose of cream was added to each Franz Cell (10 mg cream per cm² of skin tissue). The diffusion cells were maintained at a skin surface temperature of 32±1° C. After 24-hour incubation, the skin surface was cleaned with Q-tips (wet Q-tip and dry Q-tip for three cycles) to remove any surface residue of the applied test article. The skin surface was then washed with 45° C. warm water for three cycles. Skin tissues were then removed from the Franz Cell and tape stripped. The first two tape strips were discarded. The tape-stripping process was continued for an additional 15 times. The 15-tape strips were collected, quantified using liquid chromatography tandem mass spectrometry (LC/MS/MS), and labelled “stratum corneum”. Epidermis and dermis layers were separated using a scalpel. The epidermis was collected, and the lipids extracted from any remaining stratum corneum and the epidermis using baths containing chloroform/methanol mixtures. The baths were gathered, evaporated, and dissolved into an appropriate mobile phase for quantitation by HPLC/MS/MS analysis.

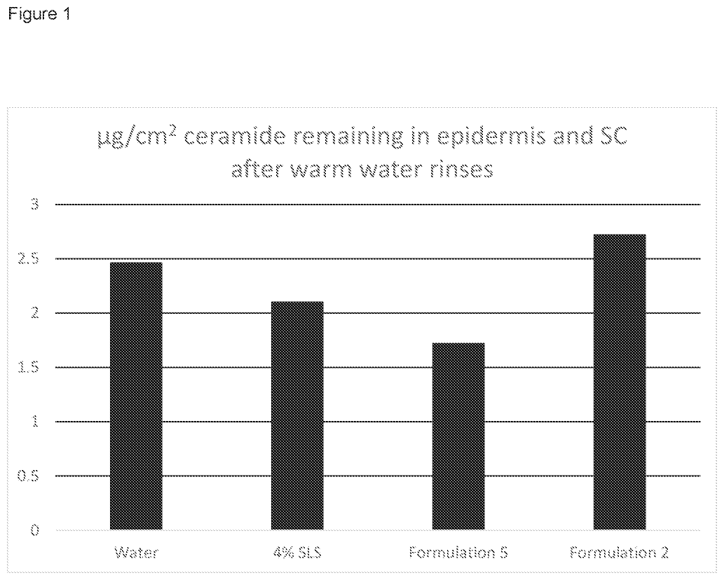

According to the literature (ref), there are twelve common ceramides in human skin. N-lignoceroyl-phytosphingosine (Ceramide NP) and N-(2′-(R)-hydroxylignoceroyl)-D-erythro-phytosphingosine (Ceramide AP) are among the most abundant ceramides in human skin. In addition to quantifying Ceramides NP and AP in this lipid extraction study, N-Lignoceroyl-D-erythro-Sphingosine (Ceramide NS) and N-lignoceroyl-D-erythro-sphinganine (Ceramide NDS) were also quantified from the tape strips and epidermal extraction baths described in this example. The total nanograms of Ceramides NP, AP, NS and NDS extracted from the samples labeled “stratum corneum” and “epidermis” after three warm (45° C.) water rinses were added together and normalized to one square centimeter of human skin. As shown in FIG. 1, treating the skin with a cream formulation containing high Krafft temperature phosphate surfactants (Formulation 2 from EXAMPLE 2) did not result in ceramide extraction. After three warm water washes the amount of ceramides remaining in Formulation 2 treated skin was the same as excised skin dosed with 5 microliters of water (inert control) 24-hours prior to the warm water washes. The skin treated with the cream containing the low Krafft temperature sodium cetostearyl sulfate surfactant (Formulation 5 from EXAMPLE 2) was most efficient in extracting ceramides from human skin. A greater quantity of ceramides were extracted from Formulation 5 treated skin after 3 warm water washes than from the 4% sodium lauryl sulfate positive control.

### Example 4

Atopic dermatitis clinical studies use the Eczema Area and Severity Index (EASI) as a validated scoring system to measure the efficacy of topically applied products. The EASI score assesses objective physician estimates of two dimensions of atopic dermatitis: 1) disease extent and 2) clinical signs. Scoring the extent of disease is accomplished by assigning a numerical score of 0 to 6 linked to the percentage of skin affected: Score of 0=0% of skin affected; score of 1=1-9% of skin affected; score of 2=10-29% skin affected; score of 3=30-49% of skin affected; score of 4=50-69% skin affected; score of 5=70-89% skin affected and score of 6=90-100% of skin affected. The disease extent score is combined with scoring of the severity of four clinical signs (erythema, induration/papulation, excoriation, and lichenification) each on a scale of 0 to 3 (0=none, absent; 1=mild; 2=moderate; 3=severe) at four body sites (head and neck, trunk, upper limbs, and lower limbs). Half scores are allowed. Each body site will have a score that ranges from 0 to 72, and the final EASI score will be obtained by averaging these four scores (using multipliers 0.2 for head and neck and upper limbs and 0.3 for trunk and lower limbs). Hence, the final EASI score will range from 0 to 72 for each time point that the patient is evaluated in the clinic. EASI scores reported as “percent change from baseline” is a standard way of clinically evaluating improvement or worsening of atopic dermatitis lesions over the time course of topical product application. As an example a 1% increase in EASI % CFB at 4 weeks of treatment would imply that on average all patients treated with this cream had their atopic dermatitis worsen. Alternatively a 55% decrease in EASI % CFB at 4 weeks of treatment would mean dramatic improvement in either disease extent or clinical signs, or typically significant improvement in both disease extent and clinical signs of atopic dermatitis lesions.

It is a drug product's ability to treat atopic dermatitis significantly better than the vehicle (the same cream formulation without the active pharmaceutical ingredient) that results in pharmaceutical product approval at the FDA. Thus, EASI scores are published for both pharmaceutical products and their vehicle control topical cream products clinically evaluated for the treatment of atopic dermatitis.

Formulation 2 from EXAMPLE 2 was dosed once daily for four weeks to 45 atopic dermatitis patients. The EASI % CFB was reduced by 55.8% for AD patients treated with this blend of high Krafft temperature surfactants (53.0° C. for ceteth-10 phosphate and 58.0° C. for dicetyl phosphate) and only one patient complained of application site burning. This is in contrast to the Elidel® vehicle formulation that had 1% increase in EASI % CFB after twice daily dosing of 136 AD patients for 4 weeks. According to the Elidel® package insert this cream vehicle formulation contains the low Krafft temperature surfactant (41° C.) sodium cetostearyl sulfate and had 17 patients complain of application site burning.

1. A method for treating an epidermal barrier with decreased function, comprising topically administering a composition comprising a high Krafft temperature surfactant, a moisturizer and water to a patient in need of such treatment, wherein said composition does not include roflumilast or a keratolytic agent.

2. The method according to claim 1, wherein said high Krafft temperature surfactant is in an amount of 0.1-20% w/w.

3. The method according to claim 1, wherein said composition is selected from the group consisting of an oil in water emulsion, a water in oil emulsion, a microemulsion or nanoemulsion, and a hydrophilic or hydrophobic ointment.

4. The method according to claim 1, wherein said composition further comprises at least one additional component selected from the group consisting of a solvent, moisturizer, polymer or thickener, antifoaming agent, preservative, antioxidant, sequestering agent, stabilizer, buffer, pH adjusting solution, skin penetration enhancer, film former, dye, pigment, aerosol forming agent and fragrance.

5. The method according to claim 1, wherein said composition has a pH of 3.5-9.0.

6. The method according to claim 1, wherein said composition comprises carriers suitable for topical administration.

7. The method according to claim 1, wherein said composition further comprises an active pharmaceutical ingredient.

8. The method according to claim 7, wherein said active pharmaceutical agent is selected from the group consisting of Anthralin, Azathioprine, Tacrolimus, Coal tar, Methotrexate, Methoxsalen, Ammonium lactate, 5-fluorouracil, Propylthouracil, 6-thioguanine, Sulfasalazine, Mycophenolate mofetil, Fumaric acid esters, Corticosteroids, Corticotropin, Vitamin D analogues, Acitretin, Tazarotene, Cyclosporine, Resorcinol, Colchicine, Adalimumab, Ustekinumab, Infliximab, antibiotics, phosphodiesterase-4 inhibitors, and combinations thereof.

9. The method according to claim 1, wherein said patient with an epidermal barrier with decreased function is suffering from eczema.

10. The method according to claim 9, wherein said patient is suffering from atopic dermatitis, contact dermatitis, and/or seborrheic dermatitis.

11. A pharmaceutical composition comprising white petrolatum, isopropyl palmitate, a high Krafft temperature surfactant, diethylene glycol monoethyl ether, methylparaben, propylparaben and water, wherein said composition does not include roflumilast or a keratolytic agent.

12. The pharmaceutical composition according to claim 11, wherein said high Krafft temperature surfactant is a mixture of cetostearyl alcohol, dicetyl phosphate, and ceteth-10 phosphate.

13. The pharmaceutical composition according to claim 12, further comprising hexylene glycol.

14. The pharmaceutical composition according to claim 11, wherein said high Krafft temperature surfactant is an anionic surfactant.

15. The pharmaceutical composition according to claim 11, wherein said high Krafft temperature surfactant has a Krafft temperature above 48° C.

16. The pharmaceutical composition according to claim 15, wherein said high Krafft temperature surfactant has a Krafft temperature above 50° C.

17. The pharmaceutical composition according to claim 16, wherein said high Krafft temperature surfactant has a Krafft temperature above 52° C.

18. The pharmaceutical composition according to claim 11, wherein said composition does not contain an active pharmaceutical ingredient.

19. A composition comprising

| | | |
|---|---|---|
| White Petrolatum | 10.0% | w/w |
| Isopropyl Palmitate | 5.0% | w/w |
| Crodafos CES | 10.0% | w/w |
| Diethylene glycol monoethyl ether (Transcutol P) | 25% | w/w |
| Methylparaben | 0.2% | w/w |
| Propylparaben | 0.05% | w/w |
| | |
|---|---|
| Purified Water | q.s. ad 100 (49.75%) |

wherein said composition does not contain an active pharmaceutical ingredient.

20. A composition comprising

| | | |
|---|---|---|
| White Petrolatum | 10.0% | w/w |
| Isopropyl Palmitate | 5.0% | w/w |
| Crodafos CES | 10.0% | w/w |
| Hexylene glycol | 2.0% | w/w |
| Diethylene glycol monoethyl ether (Transcutol P) | 25.0% | w/w |
| Methylparaben | 0.2% | w/w |
| Propylparaben | 0.05% | w/w |
| | |
|---|---|
| Purified Water | q.s. ad 100 (47.75%) |

wherein said composition does not contain an active pharmaceutical ingredient.

21. A method for reducing the extraction of epidermal lipids and increasing epidermal barrier function, comprising topically administering a formulation comprising a high Krafft temperature surfactant, a moisturizer and water to a patient in need of such treatment.

22. The method according to claim 21, wherein said high Krafft temperature surfactant is an anionic surfactant.

23. The method according to claim 21, wherein said high Krafft temperature surfactant has a Krafft temperature above 48° C.

24. The method according to claim 23, wherein said high Krafft temperature surfactant has a Krafft temperature above 50° C.

25. The method according to claim 24, wherein said high Krafft temperature surfactant has a Krafft temperature above 52° C.
